(19) United States
(12) Patent Application Publication
(10) Pub. No.: US 2009/0138088 A1
(43) Pub. Date: May 28, 2009

(54) Mobile spinal fusion implant

(76) Inventors: Robert M. Scribner, Boulder, CO (US); Robert T. Scribner, Boulder, CO (US)

Correspondence Address: RYAN KROMHOLZ & MANION, S.C., POST OFFICE BOX 26618, MILWAUKEE, WI, 53226, US

(21) Appl. No.: 12/217,478

(22) Filed: Jul. 3, 2008

Related U.S. Application Data
(60) Provisional application No. 60/958,246, filed on Jul. 3, 2007.

(51) Int. Cl. A61F 2/44; A61B 19/00

(52) U.S. Cl. 623/17.16; 623/17.11; 128/898

(57) ABSTRACT

A spinal implant device for fusing adjacent vertebra having a first base member, a second base member and a center section. The center section is designed to provide flexibility between the first base member and the second base member. A channel extends through the implant from the first base member through the center section to the second base member to allow for bone growth through the implant device.

### RELATED APPLICATIONS

This application claims the benefit of copending U.S. Provisional Patent Application Ser. No. 60/958,246, filed 3 Jul. 2007.

### BACKGROUND OF THE INVENTION

As shown in FIG. 1, the human spinal column 10 comprises a number of uniquely shaped bones, called the vertebra 12, a sacrum 14, and a coccyx 16. A human has twenty-four vertebrae 12 comprising seven cervical vertebrae 18, twelve thoracic vertebrae 20, and five lumbar vertebrae 22.

When viewed from the side, as in FIG. 1, the spinal column 10 forms a generally S-shaped curve. The curve serves to support the head, which is relatively heavy.

As FIG. 1 shows, each vertebra 12 includes a vertebral body 27, which extends on the anterior (i.e., front or chest) side of the vertebra 12. The vertebral body 27 is in the shape of an oval disk. A “cushion,” called an intervertebral disk 24, is located between adjacent vertebral bodies 27. An opening, called the vertebral foramen 26, is located on the posterior (i.e., back) side of each vertebra 12. The spinal ganglion 28 pass through the foramen 26.

Under stress, the inner material of a disk 24 may swell, pushing through its tough outer membrane. As seen in FIG. 3, the entire disk 24 may become distorted. All or part of the core material may protrude through the outer casing at a weak spot, pressing against surrounding nerves. If further activity or injury causes the membrane to rupture or tear, the disk material can injure the spinal cord or the nerves that radiate from it.

Currently available artificial discs pose several problems, including material wear and implant loosening. Because of the moving parts and associated friction of an artificial disc, the materials can break down leading to wear debris and altered implant performance. As the artificial disc experiences wear, it can lead to a loosening of the joint and changed motion.

Standard fusion procedures eliminate almost all motion at the affected level of the spine. This may lead to degeneration of adjacent levels because of increased stress placed on these discs to recover the lost motion. Therefore it is desirable to provide an implant which relies on fusion for long term stability while allowing relative motion between the fused levels.

### SUMMARY OF THE INVENTION

The invention provides devices for flexibly fusing adjacent vertebra.

One aspect of the invention provides a spinal implant device for fusing adjacent vertebrae including a body with a channel through the body.

The body may include a first base member, a second base member, and a center section. The center section may be disposed between the first base member and the second base member. The center section may be coupled to both the first base member and the second base member.

The channel may extend through each of the first base member, the second base member, and the center section.

At least one of the base members may take the form of a generally rectangular plate.

At least one of the base members may take the form of a generally circular plate.

At least one of the base members may take the form of a generally oval plate.

The first base member may be integrally formed to the center section. The second base member may be integrally formed to the center section.

The center section may take the form of a center plate disposed between the first base member and the second base member with at least one leg member. The leg member may have a first end coupled to the first base member, a second end coupled to the second base member and a center portion coupled to the center plate. The center plate may have a generally rectangular configuration.

The center section may take the form of a first planar member having a first end coupled to a first end of the first base member and a second end coupled to a second end of the second base member. The center section may further include a second planar member having a first end coupled to the second end of the first base member and a second end coupled to the first planar member. The first planar member may have at least one notch formed thereon.

The center section may take the form of a spiral member having a first end coupled to the first base member and a second end coupled to the second base member.

The center section may take the form of a tubular member having a first end coupled to the first base member and a second end coupled to the second base member.

The tubular member may have a generally cylindrical cross section. A plurality of apertures may extend through the tubular member. A plurality of ridges may be formed on the surface of the tubular member.

The tubular member may have a generally hourglass shape. The tubular member may have at least one cut out portion formed on it.

The body of the implant device maybe made of at least one selected prosthetic material. The selected prosthetic material may include polyethylene, rubber, tantalum, titanium, chrome cobalt, surgical steel, bony in-growth material, ceramic, artificial bone, polyether-ether-ketone, other polymers, or a combination thereof.

The body of the implant device is made of at least one selected prosthetic material in combination with a second resorbable material.

Another aspect of the invention provides a method of spine fusion including providing a spinal implant device, the spinal implant device having a passage therethrough, the passage being sized and configured for bonegrowth therethrough and implanting the spinal implant device between a pair of adjacent vertebrae.

The method may further include a spinal implant device may be sized and configured for relative movement between the adjacent vertebrae.

The method may further include providing the spinal implant device with temporary stiffness.

The method may further include providing the spinal implant device with temporary stiffness further comprises making at least a portion of the implant device out of a resorbable material.

### BRIEF DESCRIPTION OF THE DRAWINGS

FIG. 1 is a lateral view of a human spinal column.

FIG. 2 is a close-up lateral view of a portion of the human spinal column.

FIG. 3 is a close-up lateral view of a portion of the human spinal column with a damaged disk.

FIG. 4A is a perspective view of a spinal implant according to the present invention.

FIG. 4B is a side plan view of the spinal implant of FIG. 4A.

FIG. 4C is a side plan view of the implant of FIG. 4A showing the flexibility of the spinal implant.

FIG. 4D is a top view of the spinal implant of FIG. 4A.

FIG. 5 is a close-up lateral view of a portion of the human spinal column with the spinal implant of FIG. 4A implanted between adjacent vertebra.

FIGS. 6A to 6B are perspective and side plan views, respectively, of an alternative spinal implant.

FIGS. 7A to 7C are perspective, side plan, and top plan views, respectively, of an additional alternative spinal implant.

FIGS. 8A to 8B are perspective and side plan views, respectively, of an additional alternative spinal implant.

FIGS. 9A to 9B are perspective and side plan views, respectively, of an additional alternative spinal implant.

FIGS. 10A to 10B are perspective and side plan views, respectively, of an additional alternative spinal implant.

FIG. 11 is a perspective view of the implant of FIG. 6A including a resorbable layer.

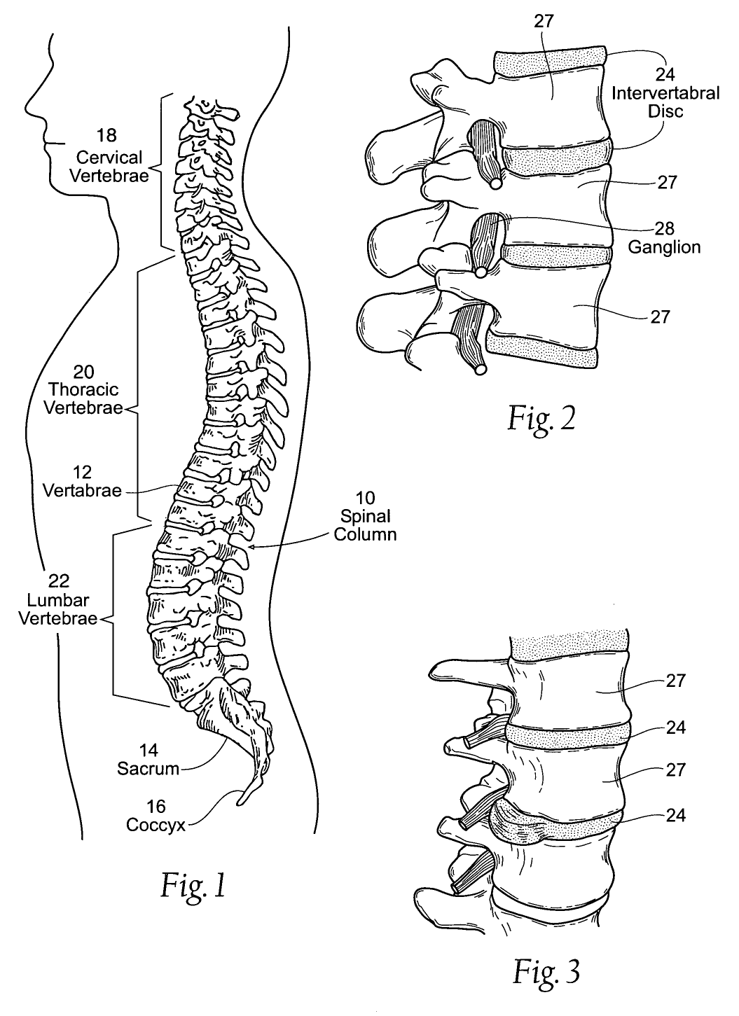

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

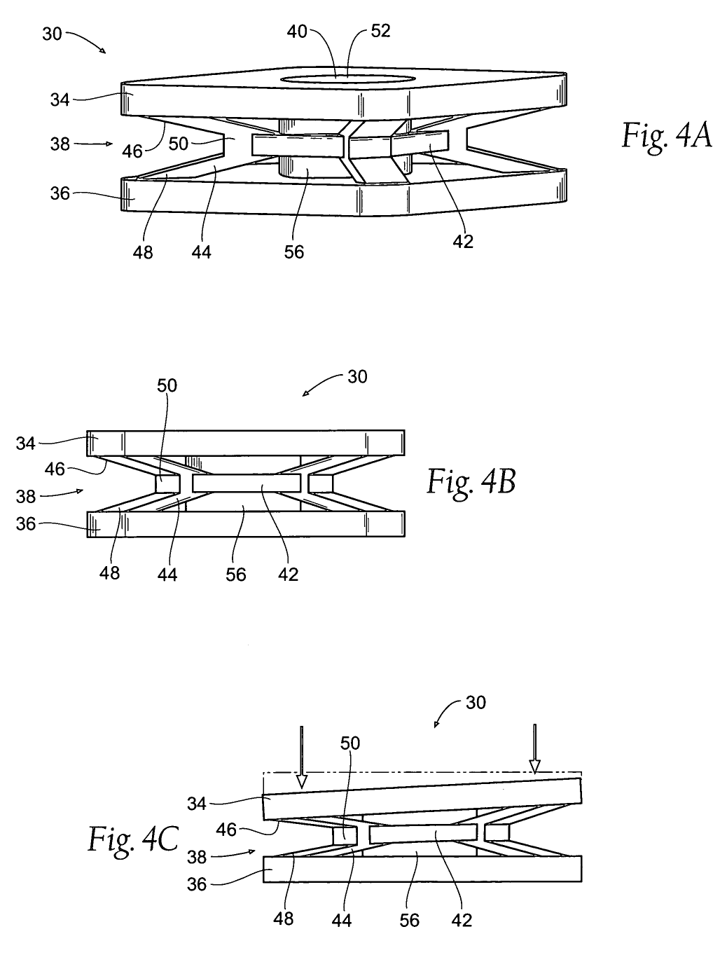

FIGS. 4A to 4D and FIGS. 6A to 10B show various illustrated embodiments of a spinal implant 30, 130, 230, 330, 430, 530. The implants 30, 130, 230, 330, 430, 530 preferably include a body 32 made up of a pair of spaced apart base members 34, 36 and a center section 38, 138, 230, 338, 438, 538 extending between the base members 34, 36. The implants 30, 130, 230, 330, 430, 530 further include a channel 40 extending through the body 32 of the implant from the first base member 34 through the center section 38, 138, 230, 338, 438, 538 to the second base member 36. It is contemplated that the base members 34, 36 may have various sizes and shapes to provide the best anatomical fit for a particular patient. It is further contemplated that the center section 38, 138, 230, 338, 438, 538 may have various configurations to allow the base members 34, 36 to move in a manner consistent with natural movement.

In the illustrated embodiment of FIGS. 4A to 4D, the center section 38 has a generally square plate 42 with a plurality of securing member 44 coupled to the square plate 42 and the pair of base members 34, 36. In the illustrated embodiment four securing members 44 are utilized, one at each corner of the square plate 42. Each securing member 44 is generally v-shaped and is coupled to a first base member 34 at the first end 46 thereof, the second base member 36 at the second end 48 thereof and the square plate 42 at the center 50 of the securing member 44.

A channel 40 preferably extends through the entire implant device 30. The channel 40 allows for bone graft and fusion through the implant device 30. Preferably each base member 34, 36 includes an aperture 52 therethrough. An aperture 54 also extends through the center section 38 and is aligned with the apertures 52 in the pair of base members 34, 36 to form a channel 40. In the illustrated embodiment a hollow portion 56 extends between the pair of base members 34, 36, the hollow portion 56 being aligned with the apertures 52 in the base members 34, 36 to form a channel 40 through the implant device 30.

In the illustrated embodiment the channel 40 is circular in cross section. However it is contemplated that the cross section could have any shape. In the illustrated embodiment the channel 40 is centered on the device 30. However, it is contemplated that the channel 40 could have any location in the device 30.

The center section 38 is preferably designed to allow movement in flexion/extension, lateral bending and axial rotation. As shown in FIG. 4C, the center section 38 of the illustrated embodiment of the implant 30 allows for flexion or extension and lateral bending through the compression or extension of the securing members 44. It is contemplated that the thickness and type of material used will determine the amount of force necessary for flexing.

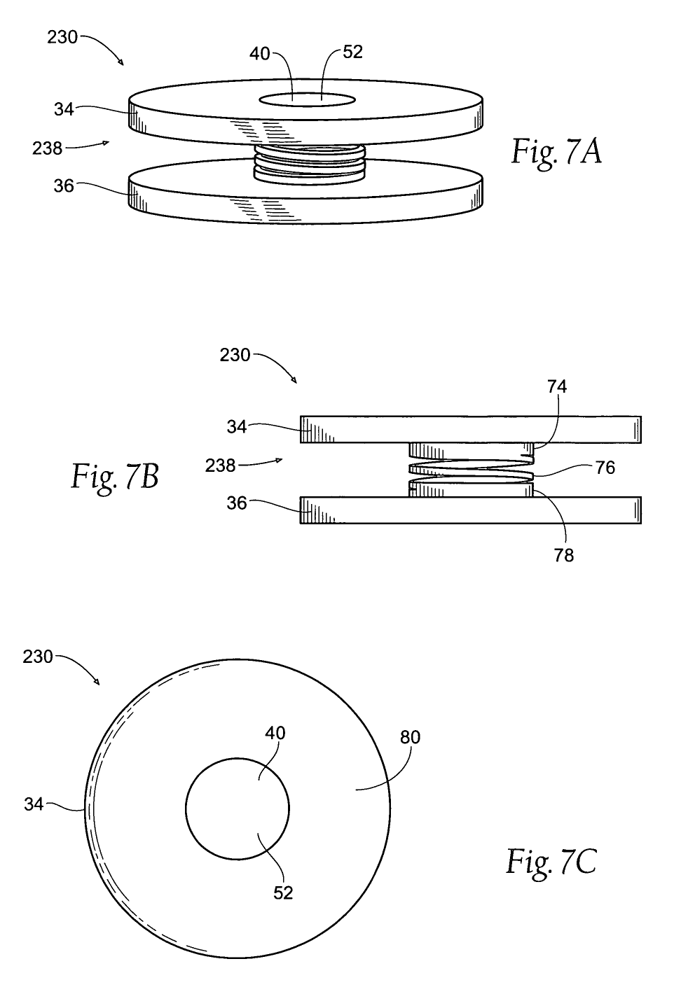

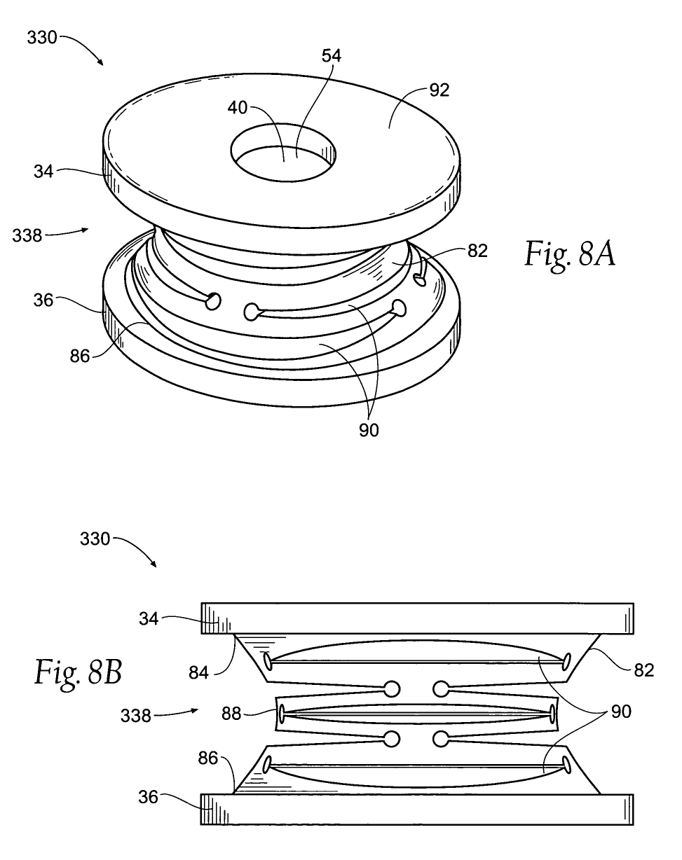

It is contemplated that the base members 34, 36 preferably comprise plates. As shown in FIG. 4D the base members of the illustrated embodiment comprise generally rectangular base plates 58. It is contemplated that the base members 34, 36 may have various configurations to provide an appropriate anatomical fit. The size and shape of the base members 34, 36 may differ according to the specific application. It is contemplated that the base members 34, 36 may take various other shapes, including, but not limited to circular (see FIG. 7C) or oval (see FIG. 8A).

FIGS. 6A and 6B show an alternative embodiment of a surgical implant 130. The embodiment shown in FIGS. 6A and 6B illustrates an alternative center section 138. The center section has a first planar member 60 extending between the two base members 34, 36. The first end 62 of the planar member 60 engages the second base member 36 near the first end 66 and engages the first base member 34 near the second end 68. Preferably, the first base member 34, second base member 36, and center section 138 are integrally formed. The first planar member 60 preferably has an aperture 54 therethrough. As described above, preferably each base member 34, 36 includes an aperture 52 therethrough. The pair of base member apertures 52 are preferably aligned with the center section aperture 54 to form a channel 40 through the implant. In the illustrated embodiment shown in FIG. 6A, the planar member 60 further includes a generally v-shaped notch 70 cut out along each side of the planar member. The center section 138 includes a second planar member 72 extending from the second end 68 of the second base member 36 to the first planar member 60 as seen in FIG. 6B.

The design of the alternative center section 138 provides for flexion/extension and lateral bending. The force necessary to bend the implant will be determined by the material properties of the implant such as elasticity of the material and thickness of the walls. In this design there is a larger area to spread the force of flexing in the flexion direction, however there is only one area of contact instead of two in the direction of flex as compared to the embodiment of FIGS. 4A to 4D. The flex in the extension direction also has one vertex of bend, although it is divided into two segments by the aperture 54 through the center.

FIGS. 7A to 7C show an additional alternative embodiment of a surgical implant 230. The embodiment shown in FIGS. 7A to 7C illustrates an alternative center section 238. The alterative center section is a generally spiral or helical member 74. The spiral member 74 is preferably hollow. The spiral member 74 is disposed between the first and second base members 34, 36 such that the first end 76 of the spiral member 74 is coupled to the first base member 34 while the second end 78 of the spiral member 74 is coupled to the second base member 36. Preferably, the first base member 34, second base member 36, and spiral member 74 are integrally formed. The spiral member 74 is designed to allow both flexing and rotation of the implant device 230. Additionally, the embodiment shown in FIGS. 7A to 7C illustrates alternative circular base members 80. As described above, preferably each circular base member 80 has an aperture 52 therethrough. The pair of base member apertures 52 are preferably aligned with the hollow portion of the spiral member 74 to form a channel 40 through the implant 230.

FIGS. 8A and 8B show an additional alternative embodiment of a surgical implant 330. The embodiment shown in FIGS. 8A and 8B illustrates an alternative center section 338. The alternative center section is a generally hourglass shaped tubular member 82. The tubular member 82 is disposed between the first and second base members 34, 36 such that the first end 84 of the tubular member 82 is coupled to the first base member 34 while the second end 86 of the tubular member 82 is coupled to the second base member 36. Preferably, the first base member 34, second base member 36, and tubular member 82 are integrally formed. As seen in FIG. 8B, the center portion 88 of the hourglass shaped tubular member 82 has a reduced diameter as compared to the end portions 84, 86 of the tubular member 82. The implant 330 preferably includes at least one cut out portion 90 formed on the hourglass shaped tubular member 82. The depth, angle, and height of cuts 90 made along the surface of the hourglass shaped tubular member 82 control how much movement there is between the first and second base members 34, 36. Additionally, the embodiment shown in FIGS. 8A and 8B illustrates alternative generally oval-shaped base members 92. As described above, preferably each oval-shaped base member 92 has an aperture 52 therethrough. The pair of base member apertures 52 are preferably aligned with the hollow portion of the tubular member 82 to form a channel 40 through the implant 330.

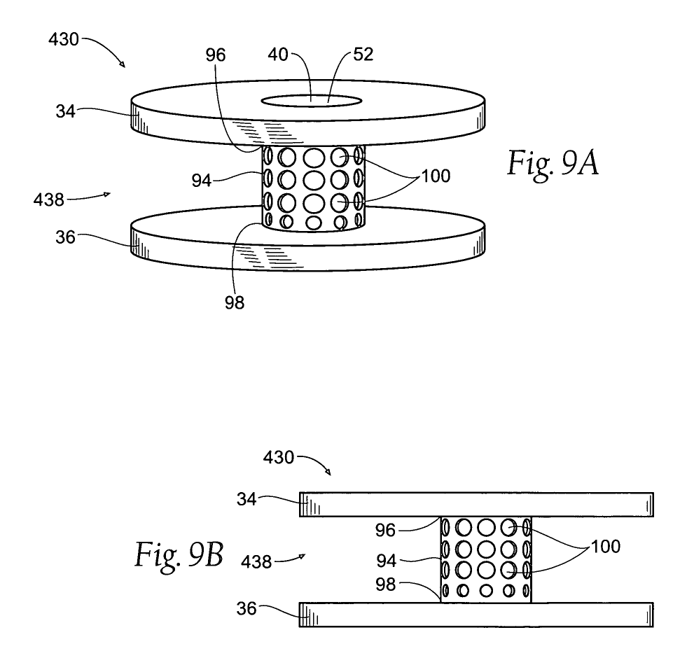

FIGS. 9A and 9B show an additional alternative embodiment of a surgical implant 430. The embodiment shown in FIGS. 9A and 9B illustrates an alternative center section 438. The alternative center section is a tubular member 94. The tubular member 94 is disposed between the first and second base members 34, 36 such that the first end 96 of the tubular member 94 is coupled to the first base member 34 while the second end 98 of the tubular member 94 is coupled to the second base member 36. Preferably, the first base member 34, second base member 36, and tubular member 94 are integrally formed. In the illustrated embodiment the tubular member 94 is generally circular in cross section, however it is contemplated alternative cross sectional configurations including but not limited to oval, may be utilized. The tubular member 94 preferably includes a plurality holes 100 formed therethrough. The holes 100 decrease the strength of the tubular member 94. It is therefore contemplated that the holes 100 may be of varying size, shape, or patterns to alter how the material flexes. As described above, preferably each base member 34, 36 has an aperture 52 therethrough. The pair of base member apertures 52 are preferably aligned with the hollow portion of the tubular member 94 to form a channel 40 through the implant 430.

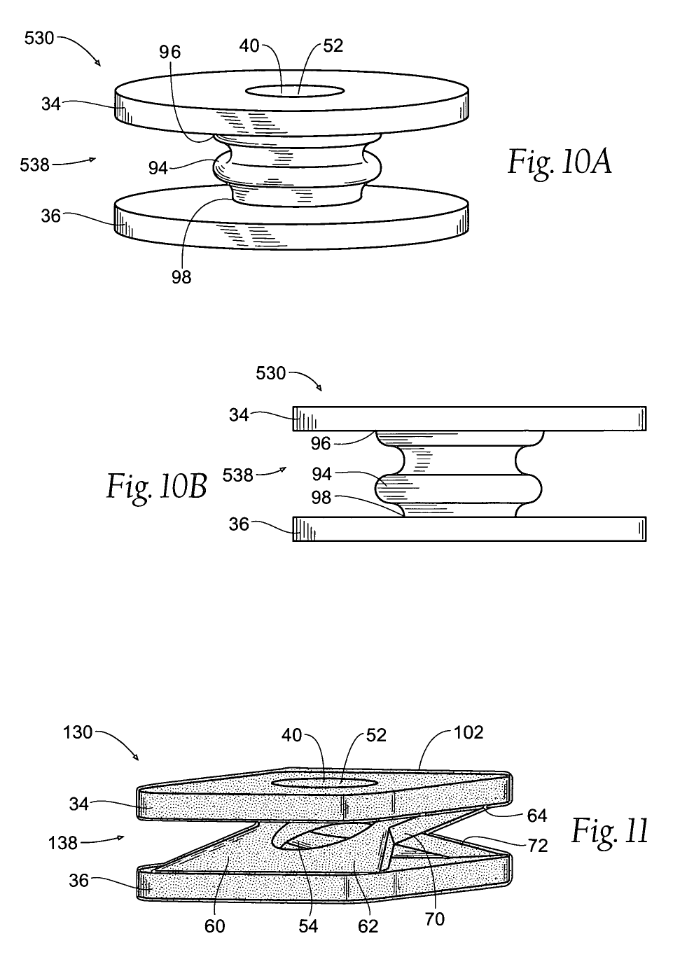

FIGS. 10A and 10B show an additional alternative embodiment of a surgical implant 530. The embodiment shown in FIGS. 10A and 10B illustrates an alternative center section 538. The alternative center section 538 is similar to the tubular member 94 of FIGS. 9A and 9B, however, the outside surface of the tubular member has a wavy configuration. In this manner the thickness of the tubular member 94 varies along the length of the tubular member 94. The tubular member 94 is disposed between the first and second base members 34, 36 such that the first end 96 of the tubular member 94 is coupled to the first base member 34 while the second end 98 of the tubular member 94 is coupled to the second base member 36. Preferably, the first base member 34, second base member 36, and tubular member 94 are integrally formed. The size of the pattern and thickness of the tubular member 94 controls the amount of force necessary and the amount of flex possible. Therefore it is contemplated that carious patters and thicknesses could be utilized to create implants with various properties. As described above, preferably each base member 34, 36 has an aperture 52 therethrough. The pair of base member apertures 52 are preferably aligned with the hollow portion of the tubular member 94 to form a channel 40 through the implant 530.

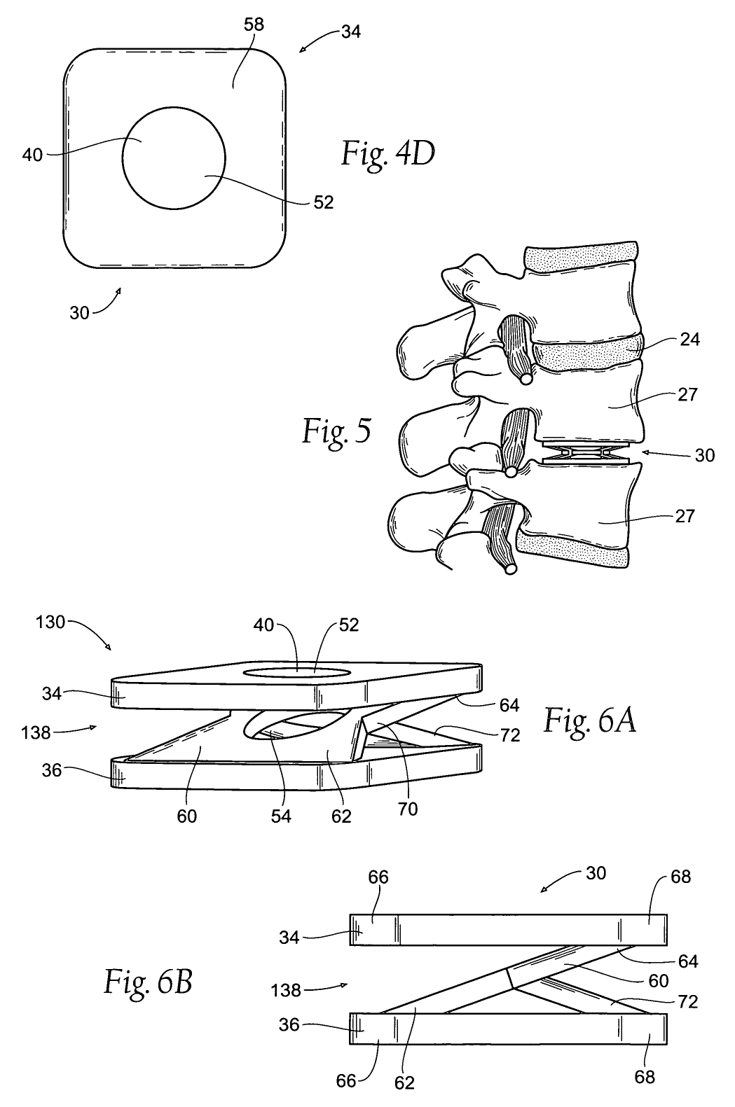

In use, the implant device 30, 130, 230, 330, 430, 530 is inserted between adjacent vertebrae 12 as shown in FIG. 5. The pair of base members 34, 36 will promote bone ingrowth to the surface of the adjacent vertebrae 12 to hold the device 30, 130, 230, 330, 430, 530 in place. Additional materials such as Hydroxyapatite may be added to the outer surface of the base members 30, 130, 230, 330, 430, 530 to increase the holding power and bone ingrowth ability on the surface of the vertebrae.

It should be understood that the illustrated embodiments allow for movement between the fused vertebrae 12 while still allowing fusion to occur. This reduces stress on the surrounding disks which would otherwise have to recover the lost movement.

It is contemplated that it may be desirable to fix the implant 30, 130, 230, 330, 430, 530 to the vertebrae 12 until fusion and bone ingrowth may occur. Any type of fixation means known in the art may be utilized to fix the implant to one or more vertebrae 12 including, but not limited to inserting screws through the implant device 30, 130, 230, 330, 430, 530 and into the adjacent vertebral bodies 27 or providing ridges along the top and bottom of the device 30, 130, 230, 330, 430, 530 to engage the vertebrae 12.

Although the illustrated embodiments show the channel 40 extending generally through the implant 30, 130, 230, 330, 430, 530 at generally the center of the base members 34, 36, it is contemplated that the channel 40 may be formed at any location on the base member 34, 36 to allow for various centers of rotation between adjacent vertebrae 12.

As stated above, the channel 40 extending through the implant 30, 130, 230, 330, 430, 530 allows for bone growth and fusion through the channel 40. In use, the channel 40 through the implant 30, 130, 230, 330, 430, 530 may be filled with allograft, autograft, or other material to assist the fusion and allow the perfusion of blood through the device 30, 130, 230, 330, 430, 530. The bones will grow onto and through the material and permanently fuse the levels. The bone growth will be limited to a known geometry within the device 30, 130, 230, 330, 430, 530, not allowing bone growth through or around the moving segments of the implant 30, 130, 230, 330, 430, 530.

Preferably the various parts of the implant 30, 130, 230, 330, 430, 530 are integrally formed. In this manner, the fit of the implant 30, 130, 230, 330, 430, 530 can not loosen because it is built as one piece. However is it also contemplated that the various parts of the implant 30, 130, 230, 330, 430, 530 may be formed separately and coupled using any means known in the art. Further, it should be understood that any base section configurations may be combined with any of the center sections.

It is contemplated that the device 30, 130, 230, 330, 430, 530 can be made of any durable prosthetic material, including, but not limited to polyethylene, rubber, a sponge material (e.g., polyethylene sponge), tantalum, titanium, chrome cobalt, surgical steel, bony in-growth material, ceramic, artificial bone, polyether-ether-ketone, other polymers, or a combination thereof.

It is further contemplated that the body of the implant device 30, 130, 230, 330, 430, 530 may include a resorbable material 102 which provides stiffness and rigidity on initial implantation of the device 30, 130, 230, 330, 430, 530, as shown in FIG. 11. Over time the resorbable material 102 may be reabsorbed by the patient's body, reducing the structural stiffness of the implant 30, 130, 230, 330, 430, 530. This resorption will occur over a period of time suitable to allow ingrowth of bone and implant fixation of the end plates 34, 36 to the vertebrae 12. This resorption will also allow for a structural column of bone to form through the center section 38, 138, 238, 338, 438, 538 of the implant 30, 130, 230, 330, 430, 530 while the implant 30, 130, 230, 330, 430, 530 has structural stiffness. On resorption of this outer component layer 102 the implant 30, 130, 230, 330, 430, 530 gains flexibility and allows motion at the spinal level. Preferably, the resorbable material may be provided as a coating or outer layer 102 formed on the implant 30, 130, 230, 330, 430, 530. The resorbable material may be of any type known in the art including, but not limited to polyglycolic acid (PLA) or polyglycolic/polylactic acid (PGLA).

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/we claim:

1. A spinal implant device for fusing adjacent vertebrae comprising:
a body sized and configured for resting between adjacent vertebrae, the body including a first base member, a second base member, and a center section, the center section being disposed between the first base member and the second base member, the center section being coupled to the first base member and the second base member;
a channel extending through the body, said channel extending through each of the first base member, the second base member and the center section.

2. A device according to claim 1 wherein at least one of said base members further comprises a generally rectangular plate.

3. A device according to claim 1 wherein at least one of said base members further comprises a generally circular plate.

4. A device according to claim 1 wherein at least one of said base members further comprises a generally oval plate.

5. A device according to claim 1 wherein said first base member is integrally formed to the center section.

6. A device according to claim 1 wherein said second base member is integrally formed to the center section.

7. A device according to claim 1 wherein said center section further comprises:
a center plate disposed between the first base member and the second base member; and
at least one leg member, said at least one leg member having a first end coupled to the first base member, a second end coupled to the second base member and a center portion coupled to the center plate.

8. A device according to claim 7 wherein said center plate has a generally rectangular configuration.

9. A device according to claim 1 wherein said center section further comprises:
a first planar member, the first planar member having a first end coupled to a first end of the first base member and a second end coupled to a second end of the second base member.

10. A device according to claim 9 further comprising a second planar member, the second planar member having a first end coupled to the second end of the first base member and a second end coupled to the first planar member.

11. A device according to claim 9 further comprising at least one notch formed in said first planar member.

12. A device according to claim 1 wherein said center section further comprises a spiral member, said spiral member having a first end coupled to the first base member and a second end coupled to the second base member.

13. A device according to claim 1 wherein said center section further comprises a tubular member, said tubular member having a first end coupled to the first base member and a second end coupled to the second base member.

14. A device according to claim 13 wherein said tubular member has a generally cylindrical cross section.

15. A device according to claim 14 further comprising a plurality of apertures extending through the tubular member.

16. A device according to claim 14 further comprising a plurality of ridges formed on the surface of the tubular member.

17. A device according to claim 13 wherein said tubular member has a generally hourglass shape.

18. A device according to claim 17 further comprising at least one cut out portion formed on the tubular member.

19. A device according to claim 1 wherein the body is made of at least one selected prosthetic material.

20. A device according to claim 19 wherein the selected prosthetic material includes polyethylene, rubber, tantalum, titanium, chrome cobalt, surgical steel, bony in-growth material, ceramic, artificial bone, or a combination thereof.

21. A device according to claim 19 wherein the body is made of at least one selected prosthetic material in combination with a second resorbable material.

22. A method of spine fusion comprising
providing a spinal implant device, said spinal implant device having a passage therethrough, the passage being sized and configured for bonegrowth therethrough; and
implanting the spinal implant device between a pair of adjacent vertebrae.

23. The method of claim 22 wherein said spinal implant device is sized and configured for relative movement between the adjacent vertebrae.

24. The method of claim 22 further comprising
providing the spinal implant device with temporary stiffness.

25. The method of claim 24 wherein providing the spinal implant device with temporary stiffness further comprises making at least a portion of the implant device out of a resorbable material.
